(19) United States
(12) Patent Application Publication
(10) Pub. No.: US 2014/0287949 A1
(43) Pub. Date: Sep. 25, 2014

(54) MULTIPLEX ASSAY FOR MEMBERS OF BINDING PAIRS

(71) Applicant: INSTITUTE FOR SYSTEMS BIOLOGY, Seattle, WA (US)

(72) Inventors: Kristin BROGAARD, Bainbridge Island, WA (US); Christopher LAUSTED, Seattle, WA (US); Leroy HOOD, Seattle, WA (US)

(73) Assignee: INSTITUTE FOR SYSTEMS BIOLOGY, Seattle, WA (US)

(21) Appl. No.: 14/213,817

(22) Filed: Mar. 14, 2014

Related U.S. Application Data
(60) Provisional application No. 61/794,207, filed on Mar. 15, 2013.
(60) Provisional application No. 61/807,670, filed on Apr. 2, 2013.

(51) Int. Cl. G01N 33/58

(52) U.S. Cl. CPC G01N 33/582
USPC 506/9; 506/16

(57) ABSTRACT

The invention provides an efficient multiplex method for identifying binding partners of small molecules and proteins. The small molecules and proteins are tagged with a nucleic acid barcode that can be used to identify the protein or small molecule, and thereby its partner.

### CROSS REFERENCE TO RELATED APPLICATION

This application claims priority from U.S. Ser. No. 61/794,207 filed Mar. 15, 2013 and U.S. Ser. No. 61/807,670 filed Apr. 2, 2013. The content of these documents is incorporated herein by reference.

### TECHNICAL FIELD

The invention is in the field of multiplex assaying. More particularly, it provides a method to identify a multiplicity of binding partners by providing complementary molecules that are tagged with oligomer identification tags or barcodes. The invention compositions and materials are particularly useful for determining mixtures of antibodies, receptors, and enzymes.

### BACKGROUND ART

Methods are currently available in the art to utilize oligomers as barcodes. This technology is described in U.S. Pat. No. 7,919,237 to NanoString™ Technologies, Inc. In this method, the oligomers that are extensions of targeting nucleotide sequences are stretched by an electrostretching technique spatially separating the monomers wherein each monomer is connected to a unique label. Thus, the pattern of labeled monomers can be used to identify the barcode on the oligomeric tag.

The present invention provides a new application for this technology whereby, for example, a multiplicity of antibodies in a sample of human serum can be identified. This provides the opportunity to diagnose autoimmune and other conditions characterized by the presence of antibodies. A multiplicity of applications for identification of protein binding partners or binding partners for small molecules is the subject of this invention.

### DISCLOSURE OF THE INVENTION

In one aspect, the invention is directed to a panel comprising a multiplicity of different molecules, each different molecule coupled to a unique oligonucleotide tag, wherein each unique oligonucleotide tag can be identified, wherein each of said different molecules binds to a complementary binding partner.

The molecules and binding partners can be proteins, such as antigens/antibodies, ligands/receptors, and enzymes/substrates, or can be small molecules.

In another aspect, the invention is directed to a method to identify a multiplicity of different binding partners each complementary to a different molecule which comprises contacting a sample to be assayed with the above-described panel so as to effect binding of any binding partners in the sample with the different tagged molecules and determining the nature of the tagged molecules using the tags as barcodes.

### BRIEF DESCRIPTION OF THE DRAWINGS

FIG. 1 is an illustration of the prior art indicating the nature of labeling of the barcode.

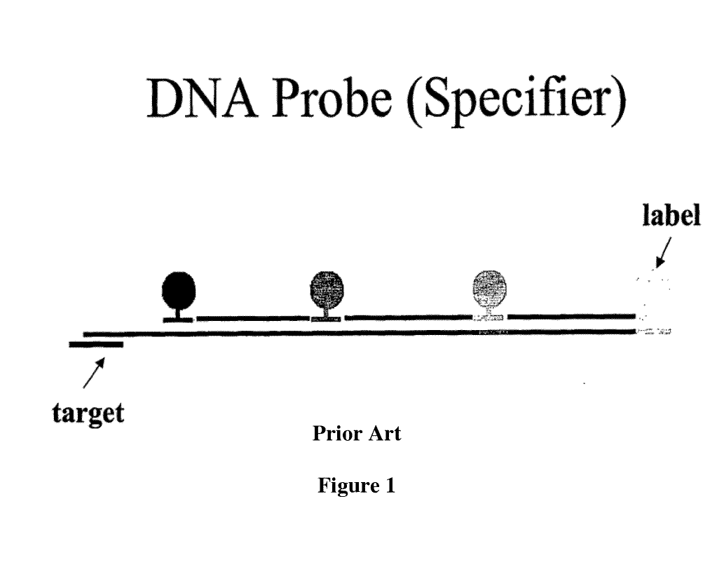

FIGS. 2A and 2B are graphs showing the ability of transferrin coupled to a 100 mer to pull down antibodies in a test sample and in a spiked control sample, respectively.

### MODES OF CARRYING OUT THE INVENTION

The invention provides efficient methods for identifying binding partners that are specific for proteins or small molecules. “Proteins” refers to amino acid sequences of any length—i.e., it includes peptides, including cyclic peptides of any molecular weight. “Proteins” also includes pseudo proteins wherein the amide linkage is replaced by an isostere such as CH₂NH, C(NH)CH₂, CSNH and the like. “Small molecules” refers to organic molecules typically having molecular weights <500 which are generally referred to by this term, for example, by the pharmaceutical industry. Specifically, “small molecules” are organic molecules that are not polymers of the same or different repeated monomeric units. Thus, for example, polyethylene glycol, nylon, and proteins are typically not considered small molecules, but nicotine, citric acid, steroids and anthocyanins are included in this definition.

For coupling the nucleic acid barcode to the different molecules in the panel, a variety of heterobifunctional or homobifunctional linkers may be used. Alternatively, the nucleic acid may be directly coupled to the small molecule or protein. In one embodiment, for coupling the tag to a protein, the oligomer tag is first coupled to 4-formyl benzamide whereby the amide groups provide coupling to the oligomer and a formyl group for reaction with succinimidyl-6-hydrazino-nicotinamide coupled to protein, for example. This results in a stable bis-aryl hydrazone linkage.

However, alternative methods are readily available in the art. Homobifunctional and heterobifunctional linkers are available commercially from Pierce Chemical Co. and well known in the art.

The barcodes themselves are nucleic acid molecules, typically comprising 90-110 nucleotides, preferably 100 nucleotides wherein in the panels of the invention, each of the oligonucleotide tags has a characteristic sequence unique to the molecule that it tags. The oligomers may be conventional DNA or RNA, or may contain modified bases, or modified sugars, or modified linkages. For example, they may contain phosphoramidate linkages, phosphothiolate linkages or constitute peptide nucleic acids. Alternative sugars include, for example, alkylated ribose at the 2-position and altered bases may also be included as long as their detectability by complementarity is preserved, or as long as they can be uniquely labeled by detectable tags. A description of labeling with multiple fluorescent tags in such barcodes is described in the above-referenced U.S. Pat. No. 7,919,237 incorporated herein by reference.

To conduct the method of the invention, the above-mentioned panel is formed by coupling each molecule (which may be a protein) in the panel with a uniquely labeled barcode. The molecules in the panel may be ligands for receptors or receptors for ligands or may be antibodies immunoreactive with antigens or antigens immunoreactive with antibodies. The panel could also include enzymes wherein substrate can be detected or substrates wherein enzymes can be detected. One specific type of panel may include, for example, proteins that are kinases.

In the method of the invention, the sample to be analyzed is contacted with the panel to effect binding of any binding partners present in the sample with their complements contained in the panel wherein each complement has been tagged differentially. Unbound tagged molecules are then removed and the molecules that have been bound to the binding partners in the sample are identified by analyzing for the presence of barcodes tagging them. The pattern of tags in the complexed binding partners is then identified by the nature of the barcodes using the labels as a guide.

The invention method provides multiplex screening, therefore of a variety of potential binding partners, including autoantibodies for detection of autoimmune diseases. This permits screening of individuals for autoantibody fingerprints, for example.

The practical applications of the multiplex assay are many. The pattern of antibodies available in serum or blood samples or fractions thereof from humans or other animals can be analyzed for the presence of infection, autoimmune disease, or conditions characterized by particular antibody patterns which can be determined empirically. Typically, in this case, the labeled compounds are proteins or other haptens specifically immunoreactive with particular antibodies. Alternatively, antibodies designed to interact with infectious agents themselves, such as viruses, may be tagged and placed on a panel to detect a microbiome. Other bodily fluids besides blood can also be used as sources of samples.

The multiplexed assay method of the invention may also be used in industrial applications such as determining patterns of fermentation or analysis for impurities in small molecule preparation and the like.

This example demonstrates that a barcoded binding partner can measure the quantity of its counterpart in a sample, in particular in a biological sample.

Briefly, samples to be tested for antibody, each having a different concentration of goat anti-transferrin antibody were incubated with an excess of anti-goat antibody labeled with biotin and with an excess or equivalent amount of transferrin coupled to a 100 nucleotide barcode oligonucleotide to form complexes. After the incubation, streptavidin derivatized beads were added and the beads, now having reacted with any biotin-derivatized complex in the sample were separated and washed to elute non-biotinylated components. The beads, now containing the anti-transferrin coupled to barcoded transferrin were eluted and bound using the barcode to bind to a barcode complement containing a series fluorescent (NanoString codesets) reporters. By counting the fluorescent reporters, the quantity of complex bound to barcode complement in each sample was determined.

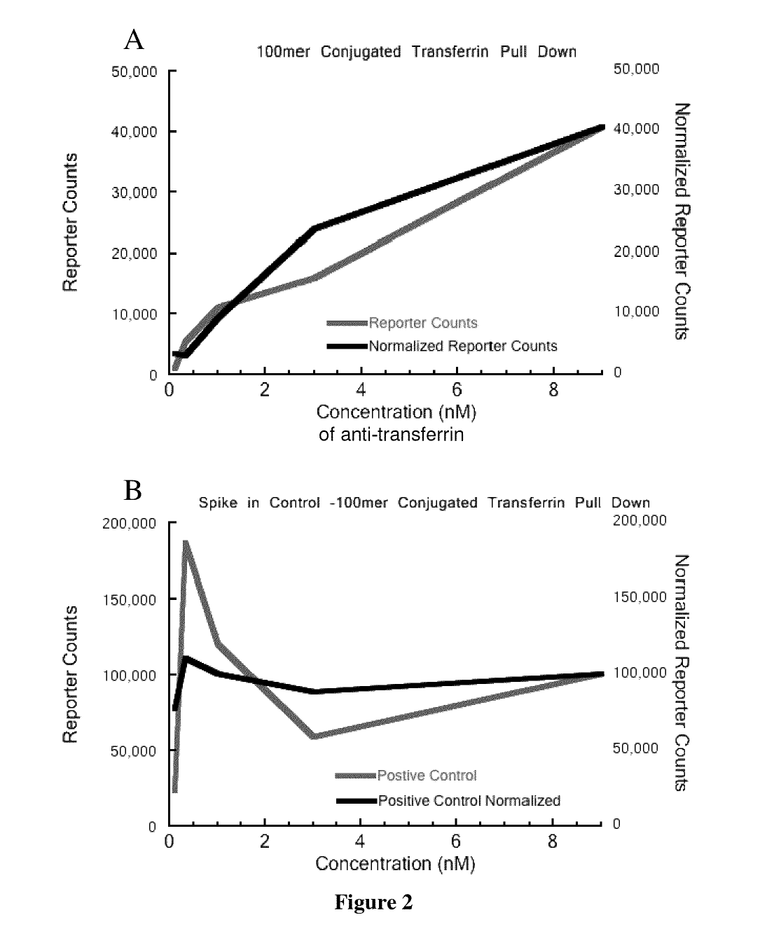

The results are shown in FIGS. 2A and 2B. The reporter counts were normalized based on the ratio between the positive controls for each sample. FIG. 2A shows that with increasing amounts of antibody, increasing amounts of reporter are obtained, thus verifying that the assay can determine quantitatively concentrations of antibody.

In FIG. 2B, results for a positive control which was added before assessment using NanoString™ protocols is presented. As noted, the normalized positive control is independent of antibody concentration.

In more detail, anti-transferrin was mixed with bovine serum albumin (BSA) (1 mg/mL), salmon sperm DNA (10 μg/mL), and Tween® (0.05%) in a PBS buffer at 6 concentrations 9 nM, 3 nM, 1 nM, 333 pM, 111 pM, and 0 pM (negative control), and the transferrin-100 mer conjugate was added to each of the 6 concentrations at a final concentration of 9 nM to each. The barcode derivatized for binding to transferrin has the structure: 5′-/5AmMC6//iSp18//iSp18/ /iSpPC/TT TAC ACC GAG TCT GGC CTG GAC GTT ATC GGA TAC GTC TCT GGA GAA AAG ACC ACT GAA GTG CTT GGT AAG GGA GGA TCG CTTACG TAC TTC ACA TTC AG -3′. 5AmMC6 is a modification that adds an amine group to the 5′ end of the oligo and required for conjugation, and iSp18 is a 18-carbon spacer used to separate the DNA oligo from the transferrin. While an iSpPC, which is a photocleavable modification that can be added internally into a DNA oligo, was included in this experiment to completely separate the DNA oligo from the transferrin, this modification was, in the end, unnecessary.

Biotinylated donkey anti-goat IgG was added to each sample at a final concentration of 15 nM, and the mixture incubated at room temperature overnight. Dynabeads® M-280 streptavidin were washed 2× with 200 μl of BSA (1 mg/mL), salmon sperm DNA (10 μg/mL), and Tween® (0.05%) in a PBS buffer and blocked for 30 minutes in the same buffer at room temperature, then resuspended in 10 μl for each sample (60 μl total). 10 μl of Dynabeads® were added to each sample and incubated with protein mixture for 30 min at room temperature. The beads were isolated and washed 4× with 400 μl of PBS+0.05% Tween®, and the complex is eluted by suspending 10 μl of 40 mM glycine (pH 2.5) and incubating at 72° C. for 30 min

Liquid was collected from beads and added to 10 μl of 5×SSPE (750 mM NaCl, 50 mM NaH₂PO₄×H₂O, 50 mM EDTA).

At this point, 10 μl of water containing 10 nM positive control 100 mer single stranded DNA oligo was added to each sample.

The control has the structure 5′/5AmMC6//ACCCACTGTGATCCTAGGCTCAAC GCATCTCAATCCCTTGAGCTCTCATTCATTATCGCAGAACGTTTGAGGAAAAGGAGG CTCGGATCGCAAAGCGTT 3′. This positive control has previously been shown to work with NanoString™ and can confirm proper hybridization and function of the NanoString™ instruments. The final pH of the sample is ˜6. Following NanoString™ standard protocols, the sample was hybridized to the codesets and processed on the NanoString™ prep station and digital analyzer. NanoString™ itself includes internal positive controls that can be used to normalize results.

1. A panel comprising a multiplicity of different tagged molecules, each different tagged molecule coupled to a unique oligonucleotide tag, wherein each unique oligonucleotide tag can be identified;
wherein each of said different tagged molecules binds to a complementary binding partner; and
wherein said tagged molecules are proteins or small molecules.

2. The panel of claim 1 wherein the tagged molecules are ligands and the binding partners are receptors for said ligands; or
wherein the tagged molecules are receptors and the tagged molecules are ligands therefor; or
wherein the tagged molecules are antigens and the binding partners are antibodies; or
wherein the tagged molecules are antibodies and the binding partners are antigens therefor; or
wherein the binding partners are enzymes and the molecules are substrates therefor; or
wherein the tagged molecules are enzymes and the binding partners are substrates therefor.

3. The panel of claim 1 wherein the oligonucleotide tags are coupled to the different molecules through linkers.

4. The panel of claim 3 wherein the linkers comprise an aryl hydrazone linkage.

5. The panel of claim 1 wherein each oligonucleotide tag is labeled with multiple fluorophores.

6. The panel of claim 1 wherein the oligonucleotide tags contain 90-110 nucleotides.

7. A method to identify a multiplicity of different binding partners, each complementary to a different molecule which comprises
(a) contacting a sample to be assayed for said binding partners with the panel of claim 1 to effect binding of any binding partners present in said sample with its complementary different tagged molecule;
(b) removing unbound tagged molecules;
(c) identifying tagged molecules that have been bound to said binding partners by identifying the tags coupled thereto.

8. The method of claim 7 wherein step (c) is preceded by separating the binding partners from the bound tagged molecules.

9. The method of claim 7 wherein the tagged molecules are ligands and the binding partners are receptors for said ligands; or
wherein the tagged molecules are receptors and the tagged molecules are ligands therefor; or
wherein the binding partners are antibodies and the tagged molecules are antigens.
wherein the tagged molecules are antibodies and the binding partners are antigens therefor; or
wherein the binding partners are enzymes and the molecules are substrates therefor; or
wherein the tagged molecules are enzymes and the binding partners are substrate therefor.

10. The method of claim 7 wherein the oligonucleotide tags are coupled to the different molecules through linkers.

11. The method of claim 7 wherein each oligonucleotide tag is labeled with multiple fluorophores.

12. The method of claims 7 wherein the oligonucleotide tags are of 90-110 nucleotides in length.


SEQUENCE LISTING (2 sequences)

SEQ ID NO: 1 - DNA, 100 bp, Artificial Sequence
  Synthetic Construct
tttacaccgagtctggcctggacgttatcggatacgtctctggagaaaagaccactgaagtgcttggtaagggaggatcgcttacgtacttcacattcag

SEQ ID NO: 2 - DNA, 99 bp, Artificial Sequence
  Synthetic Construct
acccactgtgatcctaggctcaacgcatctcaatcccttgagctctcattcattatcgcagaacgtttgaggaaaaggaggctcggatcgcaaagcgtt